(19) United States
(12) Patent Application Publication
(10) Pub. No.: US 2023/0287525 A1
(43) Pub. Date: Sep. 14, 2023

(54) METHOD FOR THE CONCENTRATION OF MICROSCOPIC SUBSTANCES DERIVED FROM LIVING ORGANISMS, ENVIRONMENTS, OR FOODS

(71) Applicant: KTEN BIO INC., Kobe (JP)

(72) Inventors: Naoyuki NISHIMURA, Hyogo (JP); Hidetomo Samori, Hyogo (JP)

(73) Assignee: KTEN BIO INC., Kobe (JP)

(21) Appl. No.: 18/182,844

(22) Filed: Mar. 13, 2023

(30) Foreign Application Priority Data
Mar. 14, 2022 (JP) 2022-060805

(51) Int. Cl. C12Q 1/70; C12Q 1/6806

(52) U.S. Cl. CPC C12Q 1/701; C12Q 1/6806; C12Q 1/6844

(57) ABSTRACT

A method for concentrating and detecting minute amounts of microscopic substances (especially pathogenic viruses) present in aqueous solutions containing biological materials such as saliva, throat wipes, and fecal suspension at three-digit microliter level volumes by adding basic substance, chelating agent, reducing agent, and protein component to PEG solutions. By combining PEG solution with these reagents and highly sensitive detection technology, it has become possible to detect and monitor microscopic substances such as viruses present in large volumes of biological samples and environmental and food materials easily, rapidly, and sensitively. As a result, it is now possible to contribute to the prevention of virus infection in the medical field and/or public and food safety field, etc.

### TECHNICAL FIELD

This invention relates to a concentration method and reagents used in the method suitable for concentrating and detecting microscopic substances (especially pathogenic viruses) derived from living organisms, environments, or foods in the field of public health and medical testing as well as in the field of general testing to ensure public safety and food safety.

### BACKGROUND ART

The microscopic substances in living organisms, environments, and foods that are the target of research and testing include viruses (especially pathogenic viruses that cause harm to animals and plants). In addition, Organelles, especially Extracellular Vesicles (EVs) including exomes, and plasmids, nucleic acids (DNA and/or RNA) and proteins in them are also the subject of research and testing, with some therapeutic applications.

In virus testing at medical sites, there are a wide variety of viruses that require vigilance, including SARS-CoV-2, which has recently spread worldwide, influenza viruses that cause a large number of infections every year, human immunodeficiency viruses that are spreading worldwide, and highly pathogenic influenza viruses and Ebola viruses. In addition, there are many viruses that have been pointed out to be at risk of large-scale infection in the future, requiring daily observation and vigilance.

On the other hand, in the food safety field, viruses that cause diarrhea in humans, such as rotaviruses, adenoviruses, astroviruses, coronaviruses and hepatitis A viruses, with noroviruses and sapoviruses at the top of the list, are among those that require special monitoring for food safety.

Furthermore, in recent years, the usefulness of detecting trace viruses in mixed samples and large volumes of environmental water for early detection of highly pathogenic virus influx from endemic areas and monitoring reemergence after an epidemic has passed has been emphasized. Thus, the development of highly sensitive systems to detect viruses (genes and/or proteins) to be monitored in large volumes of samples has become an urgent need.

The ultracentrifugal and polyethylene glycol (PEG) precipitation methods are often used to concentrate and purify microscopic substances in living organisms, environments, and foods. However, both methods require complicated and time-consuming concentration and purification procedures. Furthermore, both methods are unsuitable for mass preparation and have problems in terms of target recovery rate and foreign substance removal rate.

Therefore, we invented a simple, rapid, stable, and high recovery method for recovering viruses suspended in an aqueous solution by adding polysaccharides such as glycogen to PEG at an optimal concentration and optimizing the salt concentration to be added (hereinafter referred to as “modified PEG precipitation method”), and filed a patent application (Patent Document 1: U.S. Pat. No. 10,969,309 B2)

By the way, when the test object in the recovered concentrates is nucleic acids such as RNA or DNA, the most common method is to amplify the nucleic acids for detection, but recently, non-amplified nucleic acid detection methods have also begun to be developed.

On the other hand, when the test object is a non-nucleic acid constituent such as a protein, detection by mass spectrometry or electron microscopy is also being attempted, although the versatile antigen-antibody method is currently the most common method of detection.

The main method for amplifying and detecting nucleic acids is the PCR (Polymerase Chain Reaction) method when the detection target is DNA. And the LAMP (Loop-Mediated Isothermal Amplification) method or other methods are also used. When the detection target is RNA, the target RNA is converted to DNA (cDNA: complementary DNA) by reverse transcriptase (RT), and then amplified and detected by the DNA amplification method described above, or the RNA is amplified by Transcription Mediated Amplification (TMA) method or other methods.

In the past, the detection of amplified nucleic acids was often performed by electrophoresis or other methods after the amplified nucleic acids were once taken out.

However, with the development of the real-time (RT)-PCR and real-time (RT)-LAMP methods, which detect target products in real-time using fluorescently labeled probes or intercalation dye such as SYBR Green with a melting curve, it has become possible to amplify and detect target nucleic acids (DNA or RNA) easily, rapidly and high sensitivity with little risk of contamination.

Nevertheless, in the conventional nucleic acid amplification method, it was necessary to extract and purify the nucleic acid before conversion to cDNA by RT, and/or nucleic acid amplification in order to remove reaction inhibitors mixed in with the sample. And that procedure greatly impaired the convenience of the gene amplification method.

Therefore, the inventors independently invented the components that neutralize reaction inhibition derived from the foreign substances, and a method for direct amplification and detection without extracting and purifying nucleic acids from the sample (direct amplification method) by adding the components to the reaction solution (Patent Document No. 2: Patent 3416981 (JP), Patent Document No. 3: Patent 4735645 (JP)). Since then, this technology has been used in various detection kits, such as kits for detecting food poisoning bacteria and norovirus in stool, and SARS-CoV-2 detection kit in throat wipes and/or saliva.

However, in the direct amplification method, although samples can be added directly to the reaction solution, there is a limit to the amount of sample that can be added, and it is difficult to add more than a few μL levels. Furthermore, with the conventional amplification method after nucleic acid extraction and purification from samples, the amount of the eluent that can be added was also limited considering the amount of liquid for elution of nucleic acid, and the residual reaction inhibitors derived from the sample.

Therefore, in this study, we began to investigate a method to concentrate minute amounts of virus present in biological samples (body fluids such as saliva and throat wipes, and discharges such as feces) using the modified PEG precipitation method described above, and to detect the virus using Direct (RT-)PCR method.

First, we attempted to recover pseudo coronavirus (NATtrol SARS-CoV-2: ZeptoMetrix) added to saliva using the modified PEG precipitation method. However, the post-centrifugation sediment, which forms when using distilled water, buffers, or environmental swabs, was not formed, and a significant reduction in reaction products after Direct RT-PCR was also observed.

Therefore, we found that the modified PEG precipitation method also needs to be improved for samples that contain a large amount of foreign substances other than the targeted microscopic substances, such as biological samples.

### PRIOR ART DOCUMENTS

### Patent Documents

Patent Document 1] U.S. Pat. No. 10,969,309 B2

Patent document 2] Patent No. 3416981 (JP)

Patent document 3] Patent No. 4735645 (JP)

### SUMMARY OF INVENTION

### Problem to be Solved by the Invention

The ultracentrifugal and PEG precipitation methods are widely used for the concentration and purification of microscopic substances in living organisms, environments, and foods. However, both methods require complicated and time-consuming concentration and purification procedures. Furthermore, both methods are not suitable for mass preparation and have problems in terms of target recovery and foreign substance removal rate.

Therefore, we found that by adding polysaccharides such as glycogen to PEG at an optimal concentration and optimizing the salt concentration to be added, viruses suspended in an aqueous solution can be easily, rapidly, and stably recovered with a high recovery rate, and have filed a patent application as described above (modified PEG precipitation method).

However, when biological samples such as saliva spiked with pseudo coronavirus were used as specimens, even from the Direct real-time RT-PCR method of the sediment after the modified PEG precipitation method, non-detection, or threshold cycle (Ct) delay and/or decrease in RFU values were observed. Therefore, a significant decrease in the virus recovery rate and/or removal rate of foreign substances was suspected.

Therefore, it was necessary to develop a PEG precipitation method that can easily, quickly, and efficiently concentrate microscopic substances (especially viruses) even from biological samples that contain many foreign substances other than microscopic substances to be concentrated.

In recent years, the importance of early detection of highly pathogenic virus influx from endemic areas and surveillance of recurrent epidemics after the end of the epidemic has been emphasized. Therefore, there is an urgent need to develop a system that can efficiently concentrate viruses from large amounts of biological samples and detect them with high sensitivity.

In the Direct (RT-)PCR method, although samples can be added directly to the reaction solution, there is a limit to the amount of sample that can be added, and it is difficult to add more than a few μL levels. Furthermore, even in the method of extracting and purifying nucleic acids from microscopic substances such as viruses and then amplifying them, a certain amount of lysate is required to collect the nucleic acids. In addition, since biological samples contain a large amount of various contaminants that inhibit enzymatic reactions, the amount of sample that can be added is naturally limited when considering the effects of residues that could not be removed even by extraction and purification of nucleic acids.

### Means for Solving the Problem

By adding more effective components to the PEG solution and adjusting the precipitation condition, we will develop a PEG precipitation method that can easily, rapidly, and efficiently concentrate the target microscopic substances even from samples containing many foreign substances other than the microscopic substances. In particular, we decided to study the method that allows simple, rapid, and high and stable recovery of viruses suspended in an aqueous solution, based on the modified PEG precipitation method described in U.S. Pat. No. 10,969,309 B2.

Next, it is speculated that the components of the sample pretreatment reagent described in Patent Document 3 (Patent No. 4735645) that enable Direct RT-PCR from biological samples, may be effective in inactivating contaminants. Therefore, we investigated the possibility of combining these components with the modified PEG precipitation method using a PEG solution containing glycogen and sodium chloride (modified PEG solution).

First, the modified PEG precipitation method was performed by adding sodium hydroxide to pseudo coronavirus spiked saliva samples to basify them. As a result, a precipitate was observed in the PEG precipitation containing sodium hydroxide at a final concentration of 38.2 to 3.83 mM, but no precipitate was observed in the sodium hydroxide 1.21 mM addition group and the non-addition group. Also, in Direct real-time RT-PCR, the 12.1 mM and 3.83 mM sodium hydroxide addition groups yielded neither target virus specific PCR product nor internal control-specific product.

Based on the hypothesis that the above phenomenon is the result of the coprecipitation of many foreign substances with the target pseudovirus, by adding ethylene glycol tetraacetic acid (EGTA) as a chelating agent together with 12.1 mM sodium hydroxide, modified PEG precipitation from saliva samples spiked with pseudo coronavirus was performed. Results showed that a precipitate was observed during PEG precipitation with EGTA at final concentrations of 3.27-0.100 mM in the presence of sodium hydroxide. And the Ct and End RFU values in the Direct real-time RT-PCR also showed that precipitation of PEG with final concentrations of 1.00-0.100 mM EGTA in the presence of sodium hydroxide resulted in a significant increase of reaction products. Results similar to those obtained with EGTA were also obtained when ethylenediaminetetraacetic acid (EDTA) was used as a chelating agent.

Next, the modified PEG precipitation was then performed by adding Dithiothreitol (DTT) as a reducing agent together with sodium hydroxide and EGTA to pseudo coronavirus-spiked saliva samples. Direct real-time RT-PCR results from PEG precipitates showed increased End RFU values in groups containing 0.005-0.5 mM DTT as final concentrations compared to groups without DTT.

However, when the modified PEG precipitation method was performed by replacing saliva with distilled water (DW) and adding sodium hydroxide and EGTA as samples spiked with pseudo-coronavirus, Direct real-time RT-PCR from PEG precipitates showed that target-specific PCR products were no longer detected. However, when bovine serum albumin (BSA) was added as the protein component to the modified PEG precipitation method described above, target-specific PCR products were detected in Direct real-time RT-PCR at final BSA concentrations of 1.67-0.0167%.

A bulk saliva sample (250 μL of mixed sample from 50 individuals and 500 μL DW) spiked with 40 copies of pseudo-coronavirus were mixed with 12.1 mM sodium hydroxide, 1.00 mM EGTA, and 0.167% BSA (as final concentrations after addition of PEG solution). The PEG precipitation method was performed by mixing the above samples with an equal volume of the modified PEG solution. Direct real-time RT-PCR from the PEG precipitates showed that BSA enhanced RT-PCR even when mixed saliva was used as a sample.

Similar to the results above, Direct real-time RT-PCR detected 40 copies of pseudo coronavirus in 250 μL specimens when studies were performed using a throat swab or 10% fecal suspension instead of saliva.

The same study as [0032] was performed by replacing the detection target with viruses derived from a SARS-CoV-2 (enveloped virus) infected person and adding them to a large volume of saliva samples (250 μL of mixed sample from 50 persons), and it was possible to detect viruses equivalent to 10 copies derived from actual specimens.

The same study as [0034] was performed by replacing the detection target with a virus from a norovirus-infected person and adding them to a large-volume saliva sample (250 μl of mixed samples from 50 persons), and similar results were obtained as in [0034]. The results indicate that even when the detection target was replaced with norovirus (a non-enveloped virus), 100 copies equivalent to norovirus GI and GII can be detected.

In summary, the four elements (basic substance, chelating agent, reducing agent, and protein component), and especially the basic substance and chelating agent, are substances that can greatly improve the application range and performance of the PEG precipitation method. The newly invented elements are particularly useful for the concentration of large volume samples that contain a lot of foreign substances other than the microscopic substances to be concentrated.

Namely, the invention is a PEG precipitation method characterized in that at least basic substances and chelating agents are added individually or in a mixed state, and then the micro substances in aqueous solution are concentrated by PEG preparation. In addition, reducing agents and/or protein components can be added. The present invention also provides a method for detecting microscopic substances (particularly viruses), which comprises adding a concentrated specimen to a reaction solution without purifying nucleic acids, and directly amplifying and detecting nucleic acids in the microscopic substances.

Microorganisms include DNA viruses, RNA viruses, and retroviruses. In addition, it also includes, but is not limited to, extracellular vesicles (EVs) containing exomes, and other organelles derived from in vivo and cultured cell

Aqueous solutions that are concentrated by PEG precipitation include, but are not limited to, biological samples, environmental or food-derived solutions, as well as DW, saline, buffer solutions, and reconstituted dry materials derived therefrom.

Biological materials used as materials include bodily fluids such as blood, lymph and spinal fluid, secretions such as sweat, saliva, and swabs of the throat, nose and mouth, and excretions such as sputum, urine and feces, but not limited to these. And additionally, their dilutions with various buffers such as PBS or DW are also included.

As the method to detect the substance concentrated by the PEG precipitation method, when the detection target is DNA, there is a method of amplifying and detecting the target DNA region by the PCR method or the LAMP method. When the target is RNA, the target RNA is first converted to cDNA by RT, and then the DNA is amplified by the methods listed above. And the TMA is a method that can amplify and detect RNA templates, but it is not limited to these methods.

When the detection target is a composition other than nucleic acids, such as proteins, methods include but are not limited to, antigen-antibody methods, mass spectrometry, and the use of microscopes with high resolution, such as electron microscopes.

Of the four elements invented this time, basic substances are a general term for hydroxides of alkalimetals and alkaline earth metals, or substances such as ammonia and amines that exhibit basicity with a pH greater than 7.0 in an aqueous solution. Typical examples include, but are not limited to, sodium hydroxide, potassium hydroxide, calcium hydroxide, barium hydroxide, ammonia, copper hydroxide, and iron hydroxide. In the case of sodium hydroxide, add a final concentration of 1.21 to 38.2 mM, preferably 3.83 to 12.1 mM.

Chelating agents are substances that form complexes with metal ions to reduce their activity, and include, but are not limited to, EDTA, EGTA, NTA, DTPA, GLDA, HEDTA, GEDTA, TTHA, HIDA, DHEG, and many others. Of the above, aminocarboxylic acid chelating agents, i.e., EGTA, EDTA or their salts alone or mixtures thereof, are preferred. Aminocarboxylic acid chelating agents are added at a final concentration of 0.100 to 3.27 mM, preferably 0.317 to 1.00 mM.

In addition, reducing agents include, but are not limited to, reducing agents for protein disulfides such as DTT, BME, TCEP, and 2-Mercaptoethanol. When the reducing agent is DTT, a final concentration of 5 mM or less, preferably 0.005 to 0.5 mM, should be added.

Although BSA was used as the protein component, there is a wide variety of proteins in the world, and it is not limited to any particular one. The BSA added to the solution for concentration is 0.00167 to 1.67%, preferably 0.0167 to 0.167% in final concentration.

Of the above four elements, at least a basic substance and a chelating agent should be included, but a combination of three or all four elements may also be used. These can be added to the specimen or the PEG solution in advance or at the time of use, either singly or mixed, and there is no limitation on the elements to be added or the order of addition.

The best PEG solution to combine with the above elements is a modified PEG solution in which polysaccharides such as glycogen are added to PEG at an optimal concentration and the salt concentration to be added is also optimized, but not limited to these.

In the examples, the concentration of viruses present in various samples is exemplified. But the PEG precipitation method is widely used for the concentration of a wide variety of micro materials present in various samples, and thus it is sufficiently predictable that the present invention can be also applied to the concentration of micromaterials other than viruses in samples. Therefore, the scope of application of the invention need not be limited to viruses. For example, EVs or other organelles derived from in vivo cells or cultured cells, and plasmids, nucleic acids (DNA and/or RNA) and proteins contained in them, have recently attracted attention as targets for research and testing. There is also a movement to apply them for treatment. The invention can also provide an effective means of enrichment for these purposes.

Although the examples illustrate the concentration of viruses in 250 μL of various samples, it is possible to bring in more samples. In this case, the amount of enzymatic reaction-inhibiting substances brought along with the concentrated microscopic substances also increases. In such cases, a combination of other concentration methods such as negative charge membrane adsorption, ultrafiltration membrane, and solid precipitation is effective. And a combination with various nucleic acid extraction and purification methods, or PEG two-step concentration is also effective. These methods are particularly useful for testing viruses that exist in trace amounts in environmental water such as oceans, lakes, ponds, rivers, drinking water, and sewage.

### Effects of the Invention

We found 4 additives to dramatically improve the usefulness of the PEG precipitation method of microscopic substances derived from biological materials. 4 additives contain basic substances, chelating agents, reducing agents, and protein components. In addition, microscopic substances contain various viruses, intracellular and extracellular organelles especially Extracellular Vesicles (EVs), and plasmids, nucleic acids (DNA and/or RNA) and proteins in them. And biological materials contain body fluids such as blood, lymph fluid and spinal fluid, secretions such as sweat, saliva, and wipes from the throat, nose and mouth, and excretions such as sputum, urine and feces, and their dilutions. And they also contain cultured cells and their culture media.

Of the above four elements, at least a basic substance and a chelating agent should be included, but a combination of three or all four elements may also be used. These can be added to the specimen or the PEG solution in advance or at the time of use, either singly or mixed. In particular, the best PEG solution to combine with the above elements is a modified PEG solution in which polysaccharides such as glycogen are added to PEG at an optimal concentration and the salt concentration to be added is also optimized.

By amplifying and detecting nucleic acids in microscopic substances concentrated by this method using (RT)-PCR method, etc., it is possible to amplify and detect trace amounts of nucleic acids easily, rapidly, and with high sensitivity. Therefore, the present invention is extremely useful for detecting and monitoring viruses such as coronaviruses, influenza viruses, and noroviruses, which are highly infectious, easily transmitted through humans and the environment to humans, and cause severe symptoms, and thus contribute to the improvement of public health.

The newly invented method can withstand the enrichment of microscopic substances from samples with three-digit microliter-level volumes, and thus can also detect and screen trace amounts of nucleic acids from mixed samples. This makes it an ideal system for early detection of highly pathogenic virus influx from epidemic areas and/or monitoring of reemergence after an epidemic period has passed. In addition, by PEG two-step enrichment or combining this system with other enrichment methods such as negative charge membrane adsorption, ultrafiltration membrane, solid precipitation, as well as various nucleic acid extraction and purification methods, it will be possible to enrich microscopic substances from extremely large volumes of samples in liter units. Therefore, it is possible to contribute to the improvement of public health in a wide area by detecting or monitoring trace viruses in the environment (especially environmental water).

In recent years, EVs and other intracellular and extracellular organelles derived from in vivo cells and/or cultured cells, and the plasmids, nucleic acids (DNA and RNA) and proteins contained in them, have attracted attention as targets for research, testing, and treatment. This invention can provide an effective means for these purposes as well.

### DESCRIPTION OF THE EMBODIMENTS

### Example 1

The effect of the addition of basic substances during the concentration of pseudo coronavirus (NATtrol SARS-CoV2: ZeptoMetrix) added to biological samples by the PEG precipitation method was examined. That is, mixed saliva (250 μL: 5 μL×50 persons) and DW (500 μL), or DW (750 μL), contained with 40 copies of pseudo coronavirus was used as the sample for PEG precipitation. To sample, which contained each concentration (38.2-1.21 mM: as final concentration after adding PEG solution) of sodium hydroxide, an equal amount of 16% PEG 6,000 solution to which glycogen and sodium chloride (modified PEG solution), was added, left at room temperature for 10 minutes, and then centrifuged at 20,000 G for 10 minutes at 0° C. After the centrifugal supernatant was discarded by aspiration, a homemade sample treatment solution was added to the sediment and treated at 90° C. for 5 minutes. Next, Direct real-time RT-PCR from the heat-treated samples was performed, using homemade RT-PCR reaction solution containing RT/PCR enzymes, dNTPs, and primers/fluorescently labeled probes for detection of SARS-CoV-2 and internal control (IC). Results were assessed by comparing the presence of precipitate formation after concentration, the threshold cycle (Ct) and end RFU values after 45 cycles of PCR. Results are presented as the mean of duplicates for each group (Table 1). As a result, precipitation was observed in the PEG precipitation using sodium hydroxide at a final concentration of 38.2-3.83 mM, but no precipitation was observed in the 1.21 mM or no addition group. However, Direct real-time RT-PCR yielded no target-specific PCR products from the precipitate-forming group. Additionally, there are no PCR products including IC from the groups containing 12.1 mM and 3.83 mM sodium hydroxide. These results suggest that the addition of 12.1 mM and 3.83 mM sodium hydroxide enriched not only the target virus but also the RT-PCR inhibitors from the coexisting in the sample.

TABLE 1

| | | | Detection of | | | |
|---|---|---|---|---|---|---|
| | | Precip- | target specific | | Detection of IC | |
| | | itate | product | | specific product | |
| | | for- | Ct | RFU | Ct | RFU |
| Sample | NaOH(mM) | mation | value | value | value | value |
| Saliva | 38.2 | Yes | >45 | ND | 28.7 | 1300 |
| Saliva | 12.1 | Yes | >45 | ND | >45 | ND |
| Saliva | 3.83 | Yes | >45 | ND | >45 | ND |
| Saliva | 1.21 | No | 33.2 | 750 | 29.8 | 790 |
| Saliva | 0 | No | 34.2 | 530 | 29.5 | 750 |
| Distilled | 0 | Yes | 33.0 | 2100 | 32.0 | 1000 |
| water | | | | | | |
| Positive | — | — | 32.7 | 2150 | 32.8 | 1200 |
| Control | | | | | | |
| Negative | — | — | >45 | ND | 32.8 | 1500 |
| Control | | | | | | |

### Example 2

The effect of the addition of chelating agents during the concentration of pseudo coronavirus added to biological samples by the PEG precipitation method was examined. That is, mixed saliva (250 μL: 5 μL×50 persons) and DW (500 μL), or DW (750 μL), contained with 40 copies of pseudo coronavirus was used as the sample for PEG precipitation. To sample, which contained each concentration (10.0-0.100 mM: as final concentration after adding PEG solution) of EGTA with 12.1 mM (final concentration) sodium hydroxide, equal amounts of modified PEG solution were added, left at room temperature for 10 minutes, and then centrifuged at 20,000 G for 10 minutes at 0° C. After the centrifugal supernatant was discarded by aspiration, a homemade sample treatment solution was added to the sediment and treated at 90° C. for 5 minutes. Next, Direct real-time RT-PCR from the heat-treated samples was performed, using a homemade RT-PCR reaction solution used in Example 1. Results were assessed by comparing the presence of precipitate formation after concentration, and Ct and end RFU values after 45 cycles of PCR. Results are presented as the mean of duplicates for each group (Table 2). As a result, no precipitation was observed in PEG precipitation with 10 mM EGTA in the presence of sodium hydroxide, but it was observed in the range of 3.27 to 0.1 mM EGTA. In addition, the Ct and end RFU values in Direct real-time RT-PCR from the obtained PEG precipitates showed an increase of target-specific products from the group to which 1-0.1 mM EGTA was added with sodium hydroxide in the PEG precipitation.

TABLE 2

| | | | | Detection of target | |
|---|---|---|---|---|---|
| | | | Precipitate | specific product | |
| Sample | NaOH(mM) | EGTA(mM) | formation | Ct value | RFU value |
| Saliva | 12.1 | 10.0 | No | 33.8 | 470 |
| Saliva | 12.1 | 3.27 | Yes | 34.5 | 850 |
| Saliva | 12.1 | 1.00 | Yes | 32.9 | 1150 |
| Saliva | 12.1 | 0.317 | Yes | 33.1 | 1300 |
| Saliva | 12.1 | 0.100 | Yes | 33.8 | 1000 |
| Saliva | 12.1 | 0 | Yes | >45 | ND |
| Saliva | 0 | 0 | No | 35.0 | 300 |
| Distilled | 0 | 0 | Yes | 32.9 | 2100 |
| water | | | | | |
| Positive | — | — | — | 32.6 | 2250 |
| Control | | | | | |
| Negative | — | — | — | >45 | ND |
| Control | | | | | |

### Example 3

The effect of the addition of a chelating agent other than EGTA during the concentration of pseudo coronavirus added to biological samples by the PEG precipitation method was examined. That is, mixed saliva (250 μL: 5 μL×50 persons) and DW (500 μL), or DW (750 μL), contained with 40 copies of pseudo coronavirus was used as the sample for PEG precipitation. To sample, which contained each concentration (10.0-0.100 mM: as final concentration after adding PEG solution) of EDTA with 12.1 mM (final concentration) sodium hydroxide, equal amounts of modified PEG solution were added, left at room temperature for 10 minutes, and then centrifuged at 20,000 G for 10 minutes at 0° C. After the centrifugal supernatant was discarded by aspiration, a homemade sample treatment solution was added to the sediment and treated at 90° C. for 5 minutes. Next, Direct real-time RT-PCR from the heat-treated samples was performed, using a homemade RT-PCR reaction solution used in Example 1. Results were assessed by comparing the presence of precipitate formation after concentration, and Ct and end RFU values after 45 cycles of PCR. Results are presented as the mean of duplicates for each group (Table 3). As a result, precipitation was observed in PEG precipitation with 1.0 mM EDTA in the presence of sodium hydroxide, and the Ct and end RFU values in Direct real-time RT-PCR from the obtained PEG precipitates showed an increase of target-specific products

TABLE 3

| | | | | Detection of target | |
|---|---|---|---|---|---|
| | | | Precipitate | specific product | |
| Sample | NaOH(mM) | EGTA(mM) | formation | Ct value | RFU value |
| Saliva | 12.1 | 10.0 | Yes | 36.2 | 900 |
| Saliva | 12.1 | 1.00 | Yes | 34.4 | 1350 |
| Saliva | 12.1 | 0.100 | Yes | >45 | ND |
| Saliva | 0 | 0 | No | 33.8 | 600 |
| Distilled | 0 | 0 | Yes | 33.1 | 2350 |
| water | | | | | |
| Positive | — | — | — | 32.9 | 2000 |
| Control | | | | | |
| Negative | — | — | — | >45 | ND |
| Control | | | | | |

### Example 4

The effect of the addition of reducing agents during the concentration of pseudo coronavirus added to biological samples by the PEG precipitation method was examined. That is, mixed saliva (250 μL: 5 μL×50 persons) and DW (500 μL), or DW (750 μL), contained with 40 copies of pseudo coronavirus was used as the sample for PEG precipitation. To sample, which contained each concentration (5.00-0.005 mM: as final concentration) of dithiothreitol (DTT) with 12.1 mM (final concentration) sodium hydroxide and 1 mM (final concentration) sodium EGTA, equal amounts of modified PEG solution were added, left at room temperature for 10 minutes, and then centrifuged at 20,000 G for 10 minutes at 0° C. After the centrifugal supernatant was discarded by aspiration, a homemade sample treatment solution was added to the sediment and treated at 90° C. for 5 minutes. Next, Direct real-time RT-PCR from the heat-treated samples was performed, using a homemade RT-PCR reaction solution used in Example 1. Results were assessed by comparing the presence of precipitate formation after concentration, and Ct and end RFU values after 45 cycles of PCR. Results are presented as the mean of duplicates for each group (Table 4). As a result, end RFU values in Direct real-time RT-PCR from the obtained PEG precipitates increased in the group with 0.5-0.005 mM of DTT in the presence of sodium hydroxide and EGTA in PEG precipitation compared to the group without DTT.

TABLE 4

| | | | | | Detection of | |
|---|---|---|---|---|---|---|
| | | | | | target specific | |
| | | | | | product | |
| | | | | Precipitate | Ct | RFU |
| Sample | NaOH(mM) | EGTA(mM) | DTT(mM) | formation | value | value |
| Saliva | 12.1 | 1.00 | 5.000 | Yes | 36.4 | 500 |
| Saliva | 12.1 | 1.00 | 0.500 | Yes | 34.0 | 1450 |
| Saliva | 12.1 | 1.00 | 0.050 | Yes | 33.4 | 1600 |
| Saliva | 12.1 | 1.00 | 0.005 | Yes | 33.9 | 1400 |
| Saliva | 12.1 | 1.00 | 0 | Yes | 34.2 | 1200 |
| Saliva | 0 | 0 | 0 | No | 33.8 | 600 |
| Distilled | 0 | 0 | 0 | Yes | 33.1 | 2350 |
| water | | | | | | |
| Positive | — | — | — | — | 32.9 | 2000 |
| Control | | | | | | |
| Negative | — | — | — | — | >45 | ND |
| Control | | | | | | |

### Example 5

The effect of the addition of BSA to DW during the PEG precipitation method was examined. That is, DW (750 μL), which contained with 40 copies of pseudo coronavirus was used as the sample for PEG precipitation. To sample, which contained each concentration (1.67-0.00167%: as final concentration) of BSA with 12.1 mM (final concentration) sodium hydroxide and 1 mM (final concentration) EGTA, equal amounts of modified PEG solution were added, left at room temperature for 10 minutes, and then centrifuged at 20,000 G for 10 minutes at 0° C. After the centrifugal supernatant was discarded by aspiration, a homemade sample treatment solution was added to the sediment and treated at 90° C. for 5 minutes. Next, Direct real-time RT-PCR from the heat-treated samples was performed, using a homemade RT-PCR reaction solution used in Example 1. Results were assessed by comparing the presence of precipitate formation after concentration, and Ct and end RFU values after 45 cycles of PCR. Results are presented as the mean of duplicates for each group (Table 5). As a result, Direct real-time RT-PCR from the PEG precipitates showed that only the addition of sodium hydroxide and EGTA to the PEG solution did not generate any RT-PCR products, but the coexistence of BSA (especially 0.167-0.0167% as final concentrations) generated the products.

TABLE 5

| | | | | | Detection of | |
|---|---|---|---|---|---|---|
| | | | | | target specific | |
| | | | | | product | |
| | | | | Precipitate | Ct | RFU |
| Sample | NaOH(mM) | EGTA(mM) | BSA(%) | formation | value | value |
| Distilled | 12.1 | 1.00 | 1.67 | Yes | 34.5 | 1700 |
| water | | | | | | |
| Distilled | 12.1 | 1.00 | 0.167 | Yes | 33.4 | 2900 |
| water | | | | | | |
| Distilled | 12.1 | 1.00 | 0.0167 | Yes | 34.2 | 2400 |
| water | | | | | | |
| Distilled | 12.1 | 1.00 | 0.00167 | Yes | >45 | ND |
| water | | | | | | |
| Distilled | 12.1 | 1.00 | 0 | Yes | >45 | ND |
| water | | | | | | |
| Positive | — | — | — | — | 32.4 | 2500 |
| Control | | | | | | |
| Negative | — | — | — | — | >45 | ND |
| Control | | | | | | |

### Example 6

The effect of various additives during the concentration of pseudo coronaviruses added in mixed saliva by the PEG precipitation method was examined. That is, mixed saliva (250 μL: 5 μL×50 persons) and DW (500 μL), or DW (750 μL), contained with 40 copies of pseudo coronavirus was used as the sample for PEG precipitation. To sample, which contained 12.1 mM sodium hydroxide, 1 mM EGTA, and 0.167% BSA (as final concentration), equal amounts of modified PEG solution were added, left at room temperature for 10 minutes, and then centrifuged at 20,000 G for 10 minutes at 0° C. After the centrifugal supernatant was discarded by aspiration, a homemade sample treatment solution was added to the sediment and treated at 90° C. for 5 minutes. Next, Direct real-time RT-PCR from the heat-treated samples was performed, using a homemade RT-PCR reaction solution used in Example 1. Results were assessed by comparing the presence of precipitate formation after concentration, and Ct and end RFU values after 45 cycles of PCR. Results are presented as the mean of duplicates for each group (Table 6). The results of Direct RT-PCR from the PEG precipitates showed that even when saliva was used as a sample, the effect of BSA addition in the presence of sodium hydroxide and EGTA in PEG precipitation was seen from the end RFU values.

TABLE 6

| | | | | | Detection of | |
|---|---|---|---|---|---|---|
| | | | | | target specific | |
| | | | | | product | |
| | | | | Precipitate | Ct | RFU |
| Sample | NaOH(mM) | EGTA(mM) | BSA(%) | formation | value | value |
| Saliva | 12.1 | 1.00 | 0.167 | Yes | 32.9 | 1800 |
| Saliva | 12.1 | 1.00 | 0 | Yes | 33.1 | 1300 |
| Distilled | 12.1 | 1.00 | 0.167 | Yes | 33.5 | 2400 |
| water | | | | | | |
| Distilled | 12.1 | 1.00 | 0 | Yes | >45 | ND |
| water | | | | | | |
| Distilled | 0 | 0 | 0 | Yes | 33.2 | 2300 |
| water | | | | | | |
| Positive | — | — | — | — | 31.7 | 2500 |
| Control | | | | | | |
| Negative | — | — | — | — | >45 | ND |
| Control | | | | | | |

### Example 7

The effect of various additives on the concentration of pseudo coronaviruses added to throat wipes and fecal specimens by the PEG precipitation method was examined. That is, centrifugal supernatant (250 μL) of throat wipes or 10% fecal suspension each diluted with 2× volume of DW (500 μL), or DW (750 μL) alone mixed with 40 copies of pseudo coronavirus was used as PEG precipitation. To sample, which contained 12.1 mM sodium hydroxide, 1 mM EGTA, and 0.167% BSA (as final concentration), equal amounts of modified PEG solution were added, left at room temperature for 10 minutes, and then centrifuged at 20,000 G for 10 minutes at 0° C. After the centrifugal supernatant was discarded by aspiration, a homemade sample treatment solution was added to the sediment and treated at 90° C. for 5 minutes. Next, Direct real-time RT-PCR from the heated sample was performed, using a homemade RT-PCR reaction solution used in Example 1. Results were assessed by comparing the presence of precipitate formation after concentration, and Ct and end RFU values after 45 cycles of PCR. Results are presented as the mean of duplicates for each group (Table 6). The results of Direct real-time RT-PCR from the PEG precipitates show that even when throat wipes and fecal specimens were used as samples, PEG precipitation using PEG solution with sodium hydroxide, EGTA and BSA was able to detect 40 copies of pseudo coronavirus mixed in these samples.

TABLE 7

| | | | | | Detection of | |
|---|---|---|---|---|---|---|
| | | | | | target specific | |
| | | | | | product | |
| | | | | Precipitate | Ct | RFU |
| Sample | NaOH(mM) | EGTA(mM) | BSA(%) | formation | value | value |
| Pharyngeal | 12.1 | 1.00 | 0.167 | Yes | 33.0 | 1750 |
| swab | | | | | | |
| Fecal | 12.1 | 1.00 | 0.167 | Yes | 35.1 | 1200 |
| Distilled | 12.1 | 1.00 | 0.167 | Yes | 32.3 | 2500 |
| water | | | | | | |
| Positive | — | — | — | — | 32.9 | 2100 |
| Control | | | | | | |
| Negative | — | — | — | — | >45 | ND |
| Control | | | | | | |

### Example 8

The effect of two main additives during the concentration of coronaviruses derived from an infected person added in each of the 5 mixed saliva samples by the PEG precipitation method was examined. That is, each sample that contained mixed saliva (250 μL: 5 μL×50 persons) and approximately 10 copies of coronavirus derived from the saliva of a SARS-CoV-2 infected person was used for PEG precipitation. To samples, 500 μLDW which contained 12.1 mM sodium hydroxide and 1 mM EGTA (as final concentration), and modified PEG solution (750 μL) were added, left at room temperature for 10 minutes, and then centrifuged at 20,000 G for 10 minutes at 0° C. After the centrifugal supernatant was discarded by aspiration, a homemade sample treatment solution was added to the sediment and treated at 90° C. for 5 minutes. Next, Direct real-time RT-PCR from the heat-treated samples was performed, using a homemade RT-PCR reaction solution used in Example 1. Results were assessed by comparing the presence of precipitate formation after concentration, and Ct and end RFU values after 45 cycles of PCR. Results are presented as the mean of duplicates for each group (Table 8). The results show that even when using coronaviruses, which are enveloped viruses derived from a real sample, PEG precipitation using PEG solution with sodium hydroxide and EGTA was able to detect approximately 10 copies of coronavirus in 250 μL mixed saliva samples.

TABLE 8

| | | | | Detection of target | |
|---|---|---|---|---|---|
| | | | Precipitate | specific product | |
| Sample | NaOH(mM) | EGTA(mM) | formation | Ct value | RFU value |
| Saliva-1 | 12.1 | 1.00 | Yes | 37.1 | 640 |
| Saliva-2 | 12.1 | 1.00 | Yes | 36.2 | 680 |
| Saliva-3 | 12.1 | 1.00 | Yes | 37.4 | 640 |
| Saliva-4 | 12.1 | 1.00 | Yes | 35.8 | 920 |
| Saliva-5 | 12.1 | 1.00 | Yes | 36.1 | 820 |
| Positive | — | — | — | 34.7 | 1400 |
| Control | | | | | |
| Positive | — | — | — | 36.0 | 1200 |
| Control | | | | | |
| Negative | — | — | — | >45 | ND |
| Control | | | | | |

### Example 9

The effect of two main additives during the concentration of norovirus added in mixed saliva by the PEG precipitation method was investigated. That is, each of the 2 mixed saliva samples (250 μL: 5 μL×50 persons) and distilled water (500 μL), or distilled water (750 μL), contained with approximately 100 copies of norovirus derived from the fecal suspension of norovirus GI or GII infected person was used as the sample for PEG precipitation. To sample, which contained 12.1 mM sodium hydroxide and 1 mM EGTA (as final concentration), equal amounts of modified PEG solution were added, left at room temperature for 10 minutes, and then centrifuged at 20,000 G for 10 minutes at 0° C. After the centrifugal supernatant was discarded by aspiration, a homemade sample treatment solution was added to the sediment and treated at 90° C. for 5 minutes. Next, Direct real-time RT-PCR from the heat-treated samples was performed, using a homemade RT-PCR reaction solution containing RT/PCR enzyme, dNTPs, primers/probes for norovirus GI or GII detection, and primers/probe for internal control (IC) detection. Results were assessed by comparing the presence of precipitate formation after concentration, and Ct and end RFU values after 45 cycles of PCR. The results of norovirus GI and GII detection are shown in Table 9 and Table 10, respectively. The results show that even when using norovirus, which is a nonenveloped virus derived from a real sample, PEG precipitation using PEG solution with sodium hydroxide and EGTA was able to detect approximately 100 copies of norovirus in 250 μL mixed saliva samples.

TABLE 9

| | | | | Detection of target | |
|---|---|---|---|---|---|
| | | | Precipitate | specific product | |
| Sample | NaOH(mM) | EGTA(mM) | formation | Ct value | RFU value |
| Saliva-1 | 12.1 | 1.00 | Yes | 34.8 | 1950 |
| Saliva-2 | 12.1 | 1.00 | Yes | 32.9 | 2400 |
| Distilled | — | — | Yes | 33.8 | 2400 |
| water | | | | | |
| Distilled | — | — | Yes | 33.6 | 2400 |
| water | | | | | |
| Positive | — | — | — | 32.8 | 2400 |
| Control | | | | | |
| Positive | — | — | — | 32.4 | 2200 |
| Control | | | | | |
| Negative | — | — | — | >45 | ND |
| Control | | | | | |

TABLE 10

| | | | | Detection of target | |
|---|---|---|---|---|---|
| | | | Precipitate | specific product | |
| Sample | NaOH(mM) | EGTA(mM) | formation | Ct value | RFU value |
| Saliva-1 | 12.1 | 1.00 | Yes | 34.5 | 1100 |
| Saliva-2 | 12.1 | 1.00 | Yes | 33.5 | 1250 |
| Distilled | — | — | Yes | 32.9 | 1250 |
| water | | | | | |
| Distilled | — | — | Yes | 32.3 | 1250 |
| water | | | | | |
| Positive | — | — | — | 31.9 | 880 |
| Control | | | | | |
| Positive | — | — | — | 31.9 | 940 |
| Control | | | | | |
| Negative | — | — | — | >45 | ND |
| Control | | | | | |

1. A concentration method of microscopic substances in an aqueous solution by polyethylene glycol (PEG) precipitation comprising:
(a) adding basic substance and chelating agent individually or in the mixed state
(b) subsequently centrifuging to concentrate the microscopic substances.

2. The concentration method of microscopic substances in an aqueous solution by PEG precipitation according to claim 1,
wherein reducing agent and/or protein component are further added along with the basic substance and chelating agent.

3. The concentration method of microscopic substances in an aqueous solution by PEG precipitation according to claim 1,
wherein the microscopic substances are virus, and organelles including extracellular vesicles (EVs), and plasmids, nucleic acids (DNA and/or RNA) and proteins in them.

4. The concentration method of microscopic substances in an aqueous solution by PEG precipitation according to claim 3,
wherein the virus is an enveloped virus or a non-enveloped virus.

5. The concentration method of microscopic substances in an aqueous solution by PEG precipitation according to claim 1,
wherein the basic substance is sodium hydroxide.

6. The concentration method of microscopic substances in an aqueous solution by PEG precipitation according to claim 5,
wherein the sodium hydroxide has a final concentration of 1.21-38.2 mM.

7. The concentration method of microscopic substances in an aqueous solution by PEG precipitation according to claim 1,
wherein the chelating agent is aminocarboxylic acid chelating agent.

8. The concentration method of microscopic substances in an aqueous solution by PEG precipitation according to claim 7,
wherein the aminocarboxylic acid chelating agent is selected from among glycol ether diamine tetraacetic acid (EGTA), ethylenediaminetetraacetic acid (EDTA), their salts alone and a mixture thereof.

9. The concentration method of microscopic substances in an aqueous solution by PEG precipitation according to claim 7,
wherein the aminocarboxylic acid chelating agent has a final concentration of 0.100-3.27 mM.

10. The concentration method of microscopic substances in an aqueous solution by PEG precipitation according to claim 2,
wherein the reducing agent is dithiothreitol (DTT).

11. The concentration method of microscopic substances in an aqueous solution by PEG precipitation according to claim 10,
wherein the DTT is less than 5 mM in final concentration.

12. The concentration method of microscopic substances in an aqueous solution by PEG precipitation according to claim 2,
wherein the protein component is bovine serum albumin (BSA).

13. The concentration method of microscopic substances in an aqueous solution by PEG precipitation according to claim 12, wherein the BSA has a final concentration of 0.00167-1.67%.

14. The detection method of nucleic acids in PEG precipitate comprising:
(a) adding the sample concentrated by the method according to claim 1 to a reaction solution without purifying the nucleic acids,
(b) and directly amplifying and detecting them.
